(19) United States
(12) Patent Application Publication
(10) Pub. No.: US 2004/0147447 A1
(43) Pub. Date: Jul. 29, 2004

(54) TNFR-like protein with death domain

(75) Inventors: Jian J. Lu, Green Brook, NJ (US); Bruce C. Gomes, Wilmington, DE (US); William E. Fieles, Wilmington, DE (US)

Correspondence Address: ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/791,513

(22) Filed: Mar. 1, 2004

Related U.S. Application Data
(63) Continuation of application No. 09/733,956, filed on Jun. 1, 2001, now abandoned.
Application No. 09/733,956 is a 371 of international application No. PCT/GB99/01793, filed on Jun. 8, 1999.

(30) Foreign Application Priority Data
Jun. 12, 1998 (GB) 9812607.1

(51) Int. Cl. A61K 38/17; C07H 21/04; C07K 14/715; C07K 16/30

(52) U.S. Cl. 514/12; 530/350; 530/388.22; 536/23.5

(57) ABSTRACT

The present invention provides methods and compositions based on the identification of a Death Domain Expressing Tumor Necrosis Factor Receptor Family Homologue (DETH). The present invention further provides methods and compositions for modulating apoptosis.

### FIELD OF THE INVENTION

The present invention relates to the field of apoptosis (programmed cell death). More particularly, the present invention relates to a novel 45 kilodalton protein, Death Domain Expressing Tumor Necrosis Factor Receptor Family Homologue, referred to as DETH, nucleic acid sequences encoding the protein and uses thereof.

### BACKGROUND OF THE INVENTION

Apoptotic neuronal death appears to be an important component of a number of common neurological and psychiatric disorders (Altman, J. (1992) Programmed cell death: the paths to suicide, TINS 15:278-280; Margolis, R L et al. (1994) Programmed cell death: implications for neuropsychiatric disorders, Biol Psychiatry 35:946-956; Cotman, C W and Anderson, A J (1995) A potential role for apoptosis in neurodegeneration and Alzheimer's disease, Mol Neurobiol 10:19-45; and W. G. Tatton et al (1997) [suppl], Apoptosis in neurodegenerative disorders: potential for therapy by modifying gene transcription, J. Neural Transm 49:245-268). Some of those disorders include stroke, Alzheimer's disease, Parkinson's disease, Amyotrophic Lateral Sclerosis, hereditary retinal degenerations, glaucoma and spinal muscular. Apoptosis of glial cells may also contribute to disorders like multiple sclerosis and diabetic peripheral neuropathy. The role of apoptosis in neurodegenerative disorders has been reviewed by in W. G. Tatton et al. (1997) J. Neural. Transm. [Suppl] 49:245-268.

The phenomenon of programmed cell death, or apoptosis is known to be involved in the normal course of a wide variety of developmental processes including immune and nervous system maturation. Apoptosis also plays a role in adult tissues having high cell turnover rates. A number of different physiological signals normally activate apoptosis in these contexts, but nonphysiological insults such as irradiation and exposure to drugs such as many in current use in chemotherapy which damage DNA also can trigger apoptosis. Glucocorticoids, withdrawal of cytokines, DNA damage and signals through the antigen receptors of T or B lymphocytes can all induce apoptosis, depending on the inherent susceptibility of each individual cell type. In addition, signals through death receptors located in the plasma membrane of many cells can potently induce apoptosis. Two surface death receptors have been identified, tumor necrosis factor receptor 1 (TNF-R1) and Fas receptor (FasR).

Apoptosis has been implicated as an important cellular safeguard against tumorigenesis. Under certain conditions, cells die by apoptosis in response to high-level or deregulated expression of oncogenes. Suppression of the apoptotic program by a variety of genetic lesions may contribute to the development and progression of malignancies. This is well-illustrated by the frequent mutation of the p53 tumor suppresser gene in human tumors. Wild-type p53 is required for efficient induction of apoptosis following DNA damage and cell death induced by constitutive expression of certain oncogenes. The cytotoxicity of many commonly used chemotherapeutic agents is mediated by wild-type p53. This loss of p53 function may contribute to the clinically significant problem of drug resistant tumor cells emerging following chemotherapy regimens.

At the heart of the apoptosis program lies a family of cysteine proteases, which cleave specifically at aspartic acid (aspase activity). “Caspase” is derived from cysteine protease with aspase activity. The caspases are divided into distinct subfamilies, those related to interleukin-1 beta-converting enzyme (ICE) and those related to CED-3, a protein required for cell suicide in C. elegans. At least some of the caspases appear to act upon each other in a proteolytic cascade. Caspases are proenzymes achieving enzymatic potential only after reassembly of their active subunits.

Once the cascade of caspases have been activated, a series of degradative proteolytic events occur in the cell. Caspase-3 (CPP32) is suspected to cleave the retinoblastoma protein, thereby disrupting the cell cycle. Both caspase-3 and caspase-1 (ICE) cleave poly-ADP ribose polymerase (PARP), disrupting DNA repair, while caspase-6 (Mch-2) cleaves laminins. Interestingly, the T lymphocyte-produced death-inducing molecule granzyme B is also an aspase, and is known to cleave caspases 3, 6, 7, 8, 9 and 10 is a perform-dependent manner. Similarly, granzyme A has been reported to have ICE-like activity.

Fas receptor (FasR), tumor necrosis factor receptor-1 (TNF-R1), FADD and TRADD each contain a death domain which is capable of self-association. Death domain:death domain self-interactions are involved in transmitting signals form plasma membrane receptors to down-stream signaling events. Ligation of FasR by Fas ligand (FasL) or TNF-R1 by TNF, presumably alters the conformation of FasR or TNF-R1 death domains, respectively. Through the self-interactions of death domains FADD is able to recognize conformationally-activated FasR, while TRADD recognizes activated TNF-R1. Activation of FADD induces collaboration of its death effector domain with FLICE (caspase-8) and/or FLICE2, FLICE, in turn, is thought to cleave and activate caspase-1, while FLICE2 activates caspase-2 and cleaves PARP. Each of these activating steps directs the cell towards its own self-destruction. Activation of TRADD triggers a cascade resulting in activation and nuclear translocation of transcription factors, such as NK-KB, as well as the JUN oncogenes. The death domains of FADD and TRADD have been found to interact, indicating that cross-talk may occur between the FADD and TRADD signaling pathways.

Apoptosis is an important component of a number of common neurological and psychiatric disorders and cancers. Understanding the phenomenon of programmed cell death and providing treatments for such disorders and diseases and methods for identifying therapeutics to treat such disorders and diseases is clearly desirable.

### SUMMARY OF THE INVENTION

The present invention provides a novel protein, Death Domain Expressing Tumor Necrosis Factor Receptor Family Homologue, referred to as DETH. DETH has a role in apoptosis and appears to be a member of the TNFR1/FAS/NGFR family of receptors. Overexpression of DETH in mammalian cells induces apoptosis. DETH or variant thereof or fragment thereof may be useful in the treatment of cancer or autoimmune diseases where induction of apoptosis is desirable. DETH or variant thereof or fragment thereof is also useful for screening to identify inhibitors of apoptosis or agonists of apoptosis. Inhibitors of apoptosis would be useful, for example, in the treatment of neurodegenerative diseases where inhibition of apoptosis is desirable. Agonists of apoptosis would be useful in the treatment of cancer or autoimmune disease where induction of apoptosis is desirable.

The present invention also provides nucleic acid sequences encoding DETH, fragments thereof or variants thereof.

Another aspect of the invention provides methods of using DETH of fragment thereof to identify compounds that inhibit or induce apoptosis.

A further aspect of the invention provides methods, reagents and kits for detecting DETH or nucleic acid encoding DETH in cells, body fluids or tissues.

Yet another aspect of the invention provides pharmaceutical compositions comprising DETH or variant thereof or fragment thereof, nucleic acid encoding DETH or variant thereof or fragment thereof, or antibody specific for DETH and a pharmaceutically acceptable carrier or diluent.

These and other aspects of the present invention are described in the following detailed description.

### DETAILED DESCRIPTION OF THE INVENTION

The present invention provides a novel purified 45 kilodalton protein, Death Domain Expressing Tumor Necrosis Factor Receptor Family Homologue, referred to as DETH (SEQ ID NO:2). DETH contains a death domain or region in the intracellular carboxyl terminus followed by a fifteen amino acid transmembrance region and an amino terminal extracellular. DETH appears to be a member of the TNFR1/FAS/NGFR family of receptors. Without wishing to be bound by any particular mode or theory of action, it is presently believed that DETH acts in the early stages of apoptosis. Transient expression of DETH in human cell lines HeLa, NT2 and A549 caused cells to die of apoptosis, i.e. morphologically cells which expressed DETH protein were rounded up, and DNA in the nucleus was condensed and disintegrated.

In addition to the purified full length protein, the present invention also encompasses DETH variants. A preferred DETH variant is a purified protein having at least 80% amino acid sequence identity to the DETH amino acid sequence (SEQ ID NO: 2). More preferably such DETH variant is one having at least 90% amino acid sequence identity, most preferably such DETH variant is one having at least 95% amino acid sequence identity to the DETH amino acid sequence. Especially preferred variants are those having at least 99% amino acid sequence identity to DETH.

A variant of DETH may have an amino acid sequence that is different by one or more amino acid substitutions. The variant may have conservative changes, wherein a substituted amine acid has similar structural or chemical properties, e.g., replacement of leucine with isoleucine. More rarely, a variant may have nonconservative changes, e.g., replacement of a glycine with tryptophan. Similar minor variations may also include amino acid deletions of from one to about five amino acids or insertions of from one to about five amino acids, or both. Guidance in determining which and how many amino acid residues may be substituted, inserted or deleted without abolishing biological or immunological activity may be found using computer programs well known in the art, for example, DNAStar software;

The present invention also provides fragments of DETH. Such fragments can be of any length which is shorter than the full length protein. Fragments include portions of the full length protein capable of inducing apoptosis. Such fragments can be obtained by testing of fragments of DETH in assays such as those disclosed herein to identify those fragments capable of inducing apoptosis. Fragments can also include the various regions of DETH, such as the transmembrane region (amino acids 103-118), the death region (amino acids 168-240), the serine-proline rich region (amino acids 262-325) the extracellular region (amino acids 1-102) and the intracellular region (amino acids 121-405). Other fragments include portions of the protein capable of binding to cellular ligands, and portions of the protein capable of eliciting an immune response in a mammal. Fragments of DETH can be in a purified condition and may also show deletions, insertions or substitutions of amino acids as described herein for the full length protein.

The DETH protein may also show deletions, insertions or substitutions of amino acid residues which produce a silent change and result in a functionally equivalent DETH protein. Deliberate amino acid substitutions may be made on the basis of similarity in polarity, charge, solubility, hydrophobicity, hydrophilicity, and/or the amphipathic nature of the residues as long as the biological activity of DETH is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine; asparagine, serine, theonine phenylalanine, and tyrosine.

A deletion refers to a change in either nucleotide or amino acid sequence in which one or more nucleotides or amino acid residues, respectively, are absent.

An insertion or addition refers to change in a nucleotide or amino acid sequence which has resulted in the addition of one or more nucleotides or amino acid residues, respectively, as compared to the naturally occurring DETH.

A substitution results from the replacement of one or more nucleotides or amino acids by different nucleotides or amino acids, respectively.

The present invention also provides purified nucleic acid sequences encoding DETH, variant thereof or fragment thereof. A preferred sequence encoding DETH is shown in SEQ ID NO:1. Nucleic acid sequence as used herein refers to an oligonucleotide, nucleotide or polynucleotide and fragments or portions thereof and to DNA or RNA of genomic or synthetic origin which may be single- or double-stranded, and represent the sense or antisense strand. A nucleic acid sequence encoding DETH can be obtained from mammalian cells, preferably human cells using polymerase chain reaction techniques (PCR) and appropriate primers encompassing the 3′ and 5′ ends of the sequence encoding DETH shown in SEQ ID NO:1 with the nucleic acid in the cells serving as a template. A nucleic acid sequence encoding DETH was obtained from clone g1727750 obtained from the Merck/Washington University EST Project, St. Louis, Mo., USA. Clone g1727750 was made from mRNA of a human colon epithelial cell line. Suitable primers for PCR include 5′ primer—ttt aga tct atg aac tca aca gaa tcc aac tct tct gcc and 3′ primer—gtc gac cta cag cag gtc agg aag atg gct ata aac ag. Suitable polymerase reaction conditions are those generally found in the art and exemplified herein in the examples.

The term biologically active refers to a DETH protein having structural regulatory or biochemical functions of the naturally occurring DETH protein. Likewise immunologically active defines the capability of the natural, recombinant or synthetic DETH, or any fragment thereof, to induce a specific immune response in appropriate animals or cells and to bind with special antibodies.

The term derivative as used herein refers to the chemical modification of nucleic acid encoding DETH variant or fragment, or the encoded DETH protein, variant or fragment. Illustrative of such modifications would be replacement of hydrogen by an alkyl aryl, or amino group. A DETH derivative would encode a protein which retains biological characteristics and/or activity of natural DETH.

As used herein, the term purified refers to molecules, either nucleic acid or amino acid sequences, that are removed from their natural environment and isolated or separated from at least one other component with which they are naturally associated.

A nucleic acid sequence, or portions thereof, encoding DETH and its derivatives may be produced entirely by synthetic chemistry, after which the synthetic gene may be inserted into any of the many available DNA vectors and cell systems using reagents that are well known in the art. Moreover, synthetic chemistry may be used to introduce mutations into a sequence encoding DETH or any fragment thereof.

Also included within the scope of the present invention are nucleotide sequences that are capable of hybridizing to the nucleotide sequence of DETH (SEQ ID NO:1) under various conditions of stringency. Hybridization conditions are based on the melting temperature (Tm) of the nucleic acid binding complex or probe, as taught, for example, in Berger and Kimmer (1987, Guide to Molecular Cloning Techniques, Methods in Enzymology, Vol 152, Academic Press, San Diego Calif.) and may be used at a defined “stringency” as explained below.

Maximum stringency typically occurs at about Tm-5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. As will be understood by those of skill in that art, a maximum stringency hybridization can be used to identify or detect identical polynucleotide sequences, while an intermediate (or low) stringency hybridization can be used to identify or detect similar or related polynucleotide sequences.

The term hybridization as used herein includes any process by which a strand of nucleic acid joins with a complementary strand through base pairing (Coombs J (1994) Dictionary of Biotechnology, Stockton Press, New York, N.Y.). Amplification as carried out in the polymerase chain reaction technologies may follow the process of hybridization.

Included within the scope of the present invention are alleles of DETH. As used herein, an allele or allelic sequence is an alternative form of DETH. Alleles result from a mutation, i.e., a change in the nucleic acid sequence, and generally produce altered mRNAs or polypeptides whose structure or function may or may not be altered. Any given gene may have none, one or many allelic forms. Common mutational changes which give rise to alleles are generally ascribed to natural deletions, additions or substitutions of amino acids. Each of these types of changes may occur alone, or in combination with the others, one or more times in a given sequence.

Methods for DNA sequencing are well known in the art and employ such enzymes as the Klenow fragment of DNA polymerase I, Sequenase® (US Biochemical Corp, Cleveland Ohio)), Taq polymerase (Perkin Elmer, Norwalk Conn.), thermostable T7 polymerase (Amersham, Chicago, Ill.), or combinations of recombinant polymerases and proofreading exonucleases such as the ELONGASE Amplification System marked by Gibco BRL (Gaithersburg Md.). Preferably, the process is automated with machines such as the Hamilton Micro Lab 2200 (Hamilton, Reno Rev.), Peltier Thermal Cycler (PTC200; M J Research, Watertown Mass.) and the ABI377 DNA sequencers (Perkin Elmer).

In accordance with the present invention, nucleic acid sequences which encode DETH, fragments, fusion proteins or functional equivalents thereof may be used in recombinant DNA molecules that direct the expression of DETH in appropriate host cells. Due to the inherent degeneracy of the genetic code, other DNA sequences which encode substantially the same or a functionality equivalent amino acid sequence, may be used to clone and express DETH. As will be understood by those of skill in the art, it may be advantageous to produce DETH encoding nucleotide sequences that possess non-naturally occurring codons. Codons preferred by a particular prokaryotic or eukaryotic host can be selected, for example, to increase the rate of DETH expression or to produce recombinant RNA transcripts having desirable properties, such as a longer half-life, than transcripts produced from naturally occurring sequence.

The nucleotide sequences of the present invention can be engineered in order to alter DETH coding sequence for a variety of reasons, including but not limited to, alterations which modify the cloning, processing and/or expression of the gene product. For example, mutations may be introduced using techniques which are well known in the art, such as site-directed mutagenesis to insert new restriction sites, to alter glycosylation patterns, to change codon preference, to produce splice variants, etc.

In another embodiment of the invention, a nucleic acid sequence encoding DETH or fragment thereof DETH may be ligated to a heterologous sequence to encode a fusion protein. For example, for screening of peptide libraries for inhibitors of DETH activity, it may be useful to encode a chimeric DETH protein that is recognized by a commercially available antibody. A fusion protein may also be engineered to contain a cleavage site located between a DETH sequence and the heterologous protein sequence, so that the DETH may be cleaved and purified away from the heterologous moiety. For example, a nucleic acid sequence encoding DETH or fragment thereof or variant thereof can be ligated to a sequence encoding glutathione-S-transferase (GST) to produce a GST/DETH fusion protein.

The coding sequence of DETH could be synthesized, whole or in part, using chemical methods well known in the art (see Caruthers M H et al (1980) Nuc Acids Res Symp Ser 215-23, Horn T et al (1980) Nuc Acids Res Symp Ser 225-32, etc.). Alternatively, the protein itself could be produced using chemical methods to synthesize a DETH amino acid sequence, whole or in part. For example, peptide synthesis can be performed using various solid-phase techniques well known in the art.

DETH or fragment thereof or variant thereof can be purified by methods known in the art for purification of peptides and proteins such as preparative high performance liquid chromatography and ion exchange chromatography. The composition of the synthetic peptides may be confirmed by amino acid analysis or sequencing. Additionally, the amino acid sequence of DETH, or any part thereof, may be altered during direct synthesis and/or combined using chemical methods with sequences from other proteins, or any part thereof, to produce a variant polypeptide.

A further aspect of the present invention provides vectors comprising a nucleic acid sequence encoding DETH, variant thereof or fragment thereof In order to express a biologically active DETH, the nucleotide sequence encoding DETH or its functional equivalent, is inserted into an appropriate expression vector. Methods which are well known to those skilled in the art can be used to construct expression vectors containing a nucleic acid sequence encoding DETH, fragment or variant and appropriate transcriptional or translational controls. These methods include in vitro recombinant DNA techniques, synthetic techniques and in vivo recombination or genetic recombination. Such techniques are described in, for example, Sambrook et al (1989) Molecular Cloning, A Laboratory Manual, Cold Spring Harbor Press, Plainview N.Y. and Ausubel F M et al (1989) Current Protocols in Molecular Biology, John Wiley & Sons, New York, N.Y.

A variety of expression vector/host systems may be utilized to contain and express a DETH coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g. baculovirus); plant cell systems transfected with virus expression vectors (e.g., cauliflower mosaic virus, CaMV; tobacco mosaic virus, TMV) or transformed with bacterial expression vectors (e.g. Ti or pBR322 plasmid); or animal cell systems.

The control elements or regulatory sequences of these systems vary in their strength and specificities and are those nontranslated regions of the vector, enhancers, promoters, and 3′ untranslated regions, which interact with host cellular proteins to carry out transcription and translation. Depending on the vector system and host utilized, any number of suitable transcription and translation elements, including constitutive and inducible promoters, may be used. For example, when cloning in bacterial systems, inducible promoters such as the hybrid LacZ promoter of the Bluescript® phagemid (Stratagene, LaJolla Calif.) or pSport1 (Gibco BRL) and ptrp-lac hybrids may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are most appropriate. If it is necessary to generate a cell line that contains multiple copies of DETH, vectors based on SV40 or EBV may be used with an appropriate selectable marker.

In bacterial systems, a number of expression vectors may be selected depending upon the use intended for DETH. For example, when large quantities of DETH are needed for the induction of antibodies, vectors which direct-high level expression of fusion proteins that are readily purified may be desirable. Such vectors include, but are not limited to, the multifunctional E. Coli cloning and expression vectors such as Bluescript® (Stratagene), in which the DETH coding sequence may be ligated into the vector in frame with sequences for the amino-terminal Met and the subsequent 7 residues of β-galactosidase so that a hybrid protein is produced. pGEX vectors (Promega, Madison Wis.) may be used to express DETH or fragment or variant thereof as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase and PGH may be used. For review, see Ausubel et al (supra) and Grant et al (1987) Methods in Enzymology 153:516-544).

In cases where plant expression vectors are used, the expression of a sequence encoding DETH may be derived by any of a number of promoters. For example, viral promoters such as the 35S and 19S promoters of CaMV (Brisson et al (1984) Nature 310:511-514) may be used alone or in combination with the omega leader sequence from TMB (Takamatsu et al (1987) EMBO J 6:307-311). Alternatively, plant promoters such as the small subunit of RUBISCO (Coruzzi et al (1984) EMBO J 3:1671-1680; Broglie et al (1984) Science 224:838-843); or heat shock promoters (Winter J and Sinibaldi iR M (1991) Results Probl Cell Differ 17:85-105) may be used. These constructs can be introduced into plant cells by direct DNA transformation or pathogen mediated transfection. For reviews of such techniques, see Hobbs S or Murry L E in McGraw Hill Yearbook of Science and Technology (1992) McGraw Hill New York, N.Y., pp 191-196 or Weissbach and Weissbach (1988) Methods for Plant Molecular Biology, Academic Press, New York, N.Y., pp 421-463).

An alternative expression system which could be used to express DETH is an insect system. In one such system, Autographa californica nuclear polyhedrosis virus (AcNPV) is used as a vector to express foreign genes in Spodoptera Frugiperda cells or in Trichoplusia larvae. The DETH coding sequence may be cloned into a nonessential region of the virus, such as, the polyhedrin gene, and placed under control of the polyhedrin promoter. Successful insertion of DETH will render the polyhedrin gene inactive and produce recombinant virus lacking coat protein. The recombinant viruses are then used to infect S. frugiperda cells or Trichoplusia larvae in which DETH is expressed (Smith et al (1983) J Virol 46:584; Engelhard E K et al (1994) Proc Nat Acad Sci 91:3224-7).

In mammalian host cells, a number of viral-based expression systems may be utilized. In cases where an adenovirus is used as an expression vector, a DETH coding sequence may be ligated into an adenovirus transcription/translation complex consisting of the late promoter and tripartite leader sequence. Insertion in a nonessential E1 or E3 region of the viral genome will result in a viable virus capable of expressing DETH in infected host. In addition, transcription enhancers, such as the sarcoma virus (RSV) enhancer, may be used to increase expression in mammalian host cells.

Specific initiation signals may also be required for efficient translation of a DETH sequence. These signals include the ATG initiation codon and adjacent sequences. In cases where DETH, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to cell system in use.

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, W138, etc. have specific cellular machinery and characteristic mechanisms for such post-translation activities and may be chosen to ensure the correct modification and processing of the introduced, foreign protein.

For long-term, high-yield production of recombinant proteins, stable expression is preferred. For example, cell lines which stably express DETH may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection of systems may be used to recover transformed cell lines. These include, but are not limited to, the herpes simplex virus thymidine kinase (Wigler M et al (1977) Cell 11:233-32) and adenine phosphoribosyltransferase (Lowy I et al (1980) Cell 22:817-23) genes which can be employed in TK- or aprt cells, respectively, Also, antimetabolite, antibiotic or herbicide resistance can be used as the basis for selection; for example, dhfr which confers resistance to methotrexate (Wigler M et al 1977) Proc Natl Acad Sci 77:3567-70); apt, which confers resistance to the Acad Sci 77:3567-70); apt, which confers resistance to the aminoglycosides neomycin and G418 */cikbere0Garapin F et al (1981) J Mol Biol. 150:1-14) and als or pat, which confer resistance to chlorsulfuron and phosphiaotricin acetyltransferase, respectively (Murry, supra). Additional selectable genes which have been descried for ample, trpB which allows cells to utilize indole in place of tryptophan, or hisD, which allows cells to utilize histinol in place of histidine ( )Hartman SC and R C Mulligan (1988) Proc Natl Acad Sci 85:8047-51). Visible markers such as anthocyanins, β-glycuronidase and its substrate, GUS, and luciferase and its substrate, luciferin, can be used not only to identify trarsformants, but also to quantify the amount of transient or stable protein expression attributable to a specific vector system (Rhodes C A et al (1995) Methods Mol Biol 55:121-131).

Although the presence/absence of marker gene expression suggests that the gene of interest is also present, its presence and expression should be confirmed. For example, if the sequence employing DETH is inserted within a marker gene sequence, recombinant cells containing DETH can be identified by the absence of marker gene function. Alternatively, a marker gene in response to induction or selection usually indicates expression of the tandem DETH as well.

Alternatively, host cells which contain the coding sequence for DETH and express DETH may be identified by a variety of procedures known to those of skill in the art. These procedures include, but are not limited to, DNA-DNA or DNA-RNA hybridization an protein bioassay or immunoassay techniques which include membrane, solution, or chip based technologies for the detection and/or quantification of the nucleic acid or protein.

The presence of the DETH polynucleotide sequence can be detected by DNA-DNA or DNA-RNA hybridization or amplification using probes, portions or fragments of DETH. Nucleic acid amplification based assays involve the use of oligonucleotides or oligomers based on the DETH sequence to detect transformants containing DETH DNA or RNA. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides which can be used as a probe or amplimer.

A variety of protocols that can be used for detecting and measuring the expression of DETH, using either polyclonal or monoclonal antibodies specific for the protein are known in the art. Examples include enzyme-linked and immunosorbent assay (ELISA), radioimmunoassay (RIA) and fluorescent activated cell sorting (FACS). A two-site, monoclonal-based immunoassay utilizing monoclonal antibodies reactive to two non-interfacing epitopes on DETH is preferred, but a competitive binding assay may be employed. These and other ways, a Lasoribe, among other places, in Hampton R et al (1990, Serological Methods, a Laboratory Manual, APS Press, St. Paul Minn.) and Maddox C E et al (1983, J. Exp. Med. 158:1211).

A wide variety of labels and conjugation techniques are known by those skilled in the art and can be used in various nucleic acid and amino acid assays. Means for producing labeled hybridization or PCR probes for detecting sequences related to DETH include oligolabeling, nick translation, endlabelling or PCR amplification using a labeled nucleotide. Alternatively, the DETH sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides.

A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include those radionuclides, enzymes, fluorescent, chemiluminescent, or chromogenic agents as well as substrates, cofactors, inhibitors, and magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced as shown in U.S. Pat. No. 4,816,567 incorporated herein by reference.

Host cells transformed with DETH nucleotide sequence may be cultured under conditions suitable for the expression and recovery of the encoded protein from cell culture. The protein produced by a recombinant cell may be secreted or contained intracellularly depending on the sequence and/or the vector used. As will be understood by those of skill in the art, expression vectors containing DETH can be designed with signal sequences which direct secretion of DETH through a prokaryotic or eukaryotic cell membrane. Other recombinant constructions may join DETH to nucleotide sequence encoding a polypeptide domain which will facilitate purification of soluble proteins (Kroll D J et al (1993) DNA Cell Biol 12:441-53; of discussion of vectors infra containing fusion proteins).

DETH may also be expressed as recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor XA or enterokinase (Invitrogen, San Diego Calif.) between the purification domain and DETH is useful to facilitate purification. One such expression vector provides for expression of a fusion protein compromising an DETH and contains nucleic acid encoding 6 histidine residues followed by thioredoxin and an enterokinase cleavage site. The histidine residues facilitate purification on IMIAC (immobilized metal ion affinity chromatography as described in Porath et al (1992) Protein Expression and Purification 3:23-281) while the enterokinase cleavage site provides a means of purifying the chemokine from the fusion protein.

In addition to recombinant production, fragments of DETH may be produced by direct peptide synthesis using solid-phase techniques (cf Stewart et al (1969) Solid-Phase Peptide Synthesis, W H Freeman Co, San Francisco; Merrifield synthesis may be performed using manual techniques or by automation. Automated synthesis may be achieved, for example, using Applied Biosystems 431 A Peptide Synthesizer (Perkin Elmer, Foster City Calif.) in accordance with the instructions provided by the manufacturer. Various fragments of DETH may be chemically synthesized separately and combined using chemical methods to produce the full length molecule.

DETH-specific antibodies are useful of the diagnosis of conditions and diseases associated with expression of DETH. DETH for antibody induction does not require biological activity; however, the protein fragment, or oligopeptide must be antigenic. Peptides used to induce specific antibodies may have an amino acid sequence consisting of at least five amino acids, preferably at least 10 amino acids. They should mimic a portion of the amino acid sequence of the natural protein and may contain the entire amino acid sequence of a small, naturally occurring molecule. Short stretches of DETH amino acids may be fused with those of another protein such a keyhole limpet hemocyanin and antibody produced against the chimeric molecule. Procedures well known in the art can be used for the production of antibodies to DETH. Such antibodies include, but are not limited to, polyclonal, monoclonal, chimeric, single chain. Fab fragments and fragments produced by a Fab expression library. Neutralizing antibodies, i.e., those which inhibit diner formation, are especially preferred for diagnostics and therapeutics.

For the production of antibodies, various hosts including goats, rabbits, rats, mice, etc. may be immunized by injection with DETH or any portion, fragment or oligopeptide which retains immunogenic properties. Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parrum are potentially useful human adjuvants.

Monoclonal antibodies to DETH may be prepared using any technique which provides for the production of antibody molecules by continuous cell lines in culture. These include but are not limited to the hybridoma technique originally described by Koehler and Milstein (1975 Nature 256:495-497), the human B-cell hybridoma technique (Koshor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, New York N.Y., pp 77-96).

In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,778) can be adapted to produce DETH specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86:3833-3837), and Winter G and Milstein C (1991; Nature 349:293-299).

Antibody fragments which contain specific binding sites for DETH may also be generated. For example, such fragments include, but are not limited to, the F(ab′)2 fragments. Alternatively, Fab expression libraries may be constructed to allow rapid and easy identification of monoclonal Fab fragments with the desired specificity (Huse W D et al (1989) Science 256:1275-1281).

DETH antibodies are useful in the diagnosis of conditions or diseases in which the expression of DETH is characteristic of the condition or disease, or in assays to monitor patients being treated with DETH, an agonist of DETH or an inhibitor of DETH. Diagnostic assays for DETH include methods utilizing the antibody and a label to detect DETH in human body fluids or extracts of cells or tissues. The polypeptides and antibodies of the present invention may be used with or without modification. Frequently, the polypeptides and antibodies will be labeled by joining them, either covalently or noncovalently, with a reporter molecule. A wide variety of reporter molecules are known as described herein and known in the art.

A variety of protocols for measuring DETH or fragment or variant thereof, using either polyclonal or monoclonal antibodies specific for the respective protein are known in the art. Examples include enzyme-linked immunosorbent assay (ELISA), radioimmunoassay (RIA) and fluorescent activated cell sorting (FACS). A two-site monoclonal-based immunoassay utilizing monoclonal antibodies reactive to two non-interfering epitopes on DETH can be used as can a competitive binding assay. These assays and other immunoassays are well known in the art and can be found in standard texts such as Antibodies, A Laboratory Manual, Harlow and Lane, editors, Cold Spring Harbor Laboratory, Cold Spring Harbor, N.Y. (1988).

In order to provide a basis for diagnosis, normal or standard values for DETH expression must be established. This may be accomplished, for example, by testing body fluids or cell extracts taken from normal subjects, either animal or human, in immunoassays such as those described herein, to establish a baseline level of DETH. Standard values obtained from normal samples may then be compared with values obtained from samples from subjects known to be affected or potentially affected by a disease or condition where apoptosis is implicated or where levels of DETH are associated with the presence or absence of. The difference between the baseline levels of DETH and the levels in a subject known or potentially affected by disease establishes or assists in establishing the presence of disease state.

The present invention provides methods for detecting the presence of DETH or nucleic acid encoding DETH in a tissue or other sample. For example, a detectably labeled antibody specific for DETH, variant or fragment thereof is added to a test sample of cells, biological tissues or fluids under conditions that allow bonding to DETH in the cells, tissue or fluid. Binding of labeled antibody is then measured. The presence of detectable label in the sample indicates the presence of DETH. Similarly, the presence of nucleic acid encoding DETH, variant or fragment thereof can be detected by adding detectably labeled nucleic acid sequence encoding DETH, variant or fragment thereof or a corresponding non-coding sequence to a test sample of cells biological tissue or fluid under conditions that allow hybridization of the labeled nucleic acid to nucleic acid encoding DETH. Hybridization of the labeled nucleic acid is then measured. The presence of detectable label in the sample indicates the presence in the sample of nucleic acid encoding DETH.

For use as a reagent in such assays, DETH, variant or fragment thereof, nucleic acid encoding encoding DETH, variant or fragment thereof or antibody specific for DETH preferably incorporates a detectable label, thus providing an additional embodiment of the invention. The detectable label can be any conventional type of label and is selected in accordance with the type of assay to be performed. For example, the detectable label can comprise a radiolabel such as ¹⁴C, ¹²⁵I, or ³H, an enzyme such as peroxidase, allaline or acid phosphatase, a fluorescent label such as fluoroisothiocyanate (FITC) or rhodamine, an antibody, an antigen, a small molecule such as biotin, a paramagnetic ion, a latex particle, an electron dense particle such as ferritin or a light scattering particle such as colloidal gold. Suitable methods to detect such labels include scintillation counting, autoradiography, fluorescence measurement, calorimetric measurement or light emission measurement. Suitable assays and procedures for accomplishing such labeling and detection of the labels are well known in the art and can be found, for example, in An Introduction to Radioimmunoassays and Related Techniques: Laboratory Techniques in Biochemistry and Molecular Biology, 4th Ed., T. Chard, Elsevier Science Publishers, Amsterdam, The Netherlands, 1990; Methods in Non-Radioactive Detection, Gary C. Howard, Ed, Appleton and Lange, East Norwalk, Conn., 1993 or Radioisotopes in Biology: A Practical Approach, R. J. Slater, Ed., IRL Press at Oxford University Press, Oxford, England, 1990.

For example, an antibody specific for DETH can be labeled with ¹²⁵I according to conventional methods known in the art and used to determine the location of DETH in a tissue sample such as a sample of brain tissue or stomach tissue. The labeled antibody will bind to DETH in the tissue and the presence of the ¹²⁵I label can be detected be scintillation counting or autoradiography, thus signaling the presence in the tissue of DETH.

In those disorders where the induction of apoptosis is desirable, DETH or fragment thereof, could be used as a therapeutic to trigger apoptosis. A nucleic acid sequence encoding DETH, variant or fragment thereof, could be targeted to and expressed within a cell type to induce apoptosis. Alternatively, DETH or fragment thereof could be delivered to particular cells through a number of targeting techniques. Apoptosis is desirable in autoimmune disorders such as rheumatoid arthritis, multiple sclerosis and myasthenia gravis and cancers such as lymphomas, intestinal and breast cancers.

Another embodiment of the present invention provides methods of identifying inhibitors or inducers of apoptosis, which compounds can then be used as therapeutics. DETH or fragment thereof or variant thereof can be used to screen for compounds such as small organic molecules, peptides and other types of molecules that induce apoptosis, which compounds can then be used as therapeutics to induce apoptosis. For example, a test compound can be added to cells expressing low levels of DETH or fragment thereof and control cells. Compounds that induce the death of only the DETH-expressing cells would thus be inducers of apoptosis. Such compounds could then be used in the treatment of disorders were apoptosis is desirable, such as autoimmune disorders such as rheumatoid arthritis, multiple sclerosis, myasthenia gravis and cancers such as lymphomus, intestinal and breast cancer. Thus, the present invention provides a method for identifying compounds that induce apoptosis comprising the steps of adding a test compound to cells transformed to express how levels of DETH, variant, or fragment thereof and determining whether the cells live or die. If the test compound induces cell death by apoptosis, it can be used as a therapeutic as discussed above. The morphological changes in cells which are associated with apoptosis are well known and can be used to determine the status of the cells.

In those disorders where apoptosis is not desirable, such as stroke, Alzheimer's disease ALS, spinal muscular atrophy, Huntington's Chorea and Parkinson's Disease, an inhibitor of DETH could be used as a therapeutic to inhibit apoptosis. Inhibitors of DETH can be obtained through screening mammalian cells that transiently over express DETH, variant or fragment thereof. A test compound is then added to such cells whereby when the test compound inhibits apoptosis, the cells survive. Thus, the present invention provides a method for identifying compounds that inhibit apoptosis comprising adding a test compound to cells transformed to overexpress DETH, fragment or variant thereof and determining whether the cells live or die. If the test compound inhibits apoptosis, the cells will live. If the compound is not effective in inhibiting apoptosis, the cells will die.

The DETH protein can be used to identify cellular or other types of ligands that bind to the protein. For example, DETH or fragment thereof can be used to make protein affinity columns to trap proteins or other types of ligands present in cell lysate. The proteins eluting from the affinity column can be analyzed to identify the protein or other ligand and its function.

This invention also contemplates the use of competitive drug screening assays in which neutralizing antibodies capable of binding DETH specifically compete with a test compound for binding DETH. In this manner, the antibodies can be used to detect the presence of any peptide which shares one or more antigenic determinants with DETH.

A polynucleotide, DETH, or any part thereof, may be used for diagnostic and/or therapeutic purposes. For diagnostic purposes, the DETH of this invention may be used to detect and quantitate gene expression in biopsied tissues in which expression of DETH may be implicated. The diagnostic assay is useful to distinguish between absence, presence, and excess expression of DETH and to monitor regulation of DETH levels during therapeutic intervention. Included in the scope of the invention are oligonucleotide sequences, antisense RNA and DNA molecules, and PNAs.

The present invention provides pharmaceutical compositions comprising nucleic acid sequences, proteins, antibodies, agonists, or inhibitors, alone or in combination with at least one other agent, such as stabilizing compound, and a pharmaceutically acceptable carrier or diluent. Suitable carriers and diluents include saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

Administration of pharmaceutical compositions is accomplished by any suitable route, such as orally or parenterally. Methods of parenteral delivery include topical, intra-arterial (directly to the tumor), intramuscular, subcutaneous, intramedullary, intrathecal, intraventricular, intravenous, intraperitoneal, or intranasal administration. Further details on techniques for formulation and administration may be found in the latest edition of “Remington's Pharmaceutical Sciences” (Maack Publishing Co., Exton Pa.))

Pharmaceutical compositions suitable for use in the present invention include compositions wherein the active ingredients are contained in an effective amount to achieve the intended purpose. The determination of an effective dose is well within the capability of those skilled in the art.

For any compound, the-therapeutically effective dose can be estimated initially either in cell culture assays, e.g., of neoplastic cells, or in animal models, usually mice, rabbits, dogs or pigs. The animal model is also used to achieve desirable concentration range and route of administration. Such information can then be used to determine useful doses and routes for administration in humans.

A therapeutically effective dose refers to that amount of protein or its antibodies, antagonists, or inhibitors which ameliorate the symptoms or-condition. Therapeutical efficacy and toxicity of such compounds can be determined by standard pharmaceutical procedures in cell cultures or experimental animals, e.g. ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state, e.g., tumor size and location; age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature.

Another aspect of the subject invention is to provide for hybridization or PCR probes which are capable of detecting polynucleotide sequences, including genomic sequences, encoding DETH or closely related molecules. The specificity of the probe, whether it is made from a highly specific region, e.g., 10 unique nucleotides in the 5′ regulatory region, or a less specific region, e.g. especially in the 3′ region, and the stringency of the hybridization or amplification (maximal, high, intermediate or low) will determine whether the probe identifies only naturally occurring DETH, alleles or related sequences.

It will be appreciated by those skilled in the art that as a result of the degeneracy of the genetic code, a multitude of DETH-encoding nucleotide sequences, some bearing minimal homology to the nucleotide sequences of any known and naturally occurring gene may be produced. The invention has specifically contemplated each and every possible variation of nucleotide sequence that could be made by selecting combinations based on possible codon choices. These combinations are made in accordance with the standard triplet genetic code is applied to the nucleotide sequence of naturally occurring DETH, and all such variations are to be considered as being specifically disclosed.

Probes may also be used for the detection of related inhibitor encoding sequences and should preferably contain at least 50% of the nucleotides from any of the DETH encoding sequences. The hybridization probes of the subject invention may be derived from the nucleotide sequence of SEQ. ID NO:1 or from genomic sequence including promoter, enhancer elements and introns of the naturally occurring DETH. Hybridization probes may be labeled by a variety of reporter groups, including radionuclides such as ³²P or ³⁵S, or enzymatic labels such as alkaline phosphatase coupled to the probe via avidin/biotin coupling systems, and the like.

Other means for producing specific hybridization probes for DETH DNAs include the cloning of nucleic acid sequences encoding DETH or DETH derivatives into vectors for the production of mRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides.

Nucleic acid sequences encoding DETH may be used for the diagnosis of conditions or diseases with which the expression of DETH-is associated. For example, polynucleotide sequences encoding DETH may be used in hybridization or PCR assays of fluids or tissues from biopsies to detect DETH expression. The form of such qualitative or quantitative methods may include Southern or northern analysis, dot blot or other membrane-based technologies; PCR technologies; dip stick, pin, chip and ELISA technologies. All of these techniques are well known in the art and are the basis of many commercially available diagnostic kits.

### EXAMPLES

### Example 1

### DNA Encoding DETH

I. The novel protein DETH was identified after the assembly of expressed sequence tags (EST) sequences from a proprietary database base (Lifeseq data base, Incyte, Palo Alto, Calif.) and the public Merck/Washington University EST sequence database. The initial identification of these ESTs was performed by basic local alignment search tool (BLAST) analysis of the databases using the death domain sequence of RIP (TNF receptor interacting protein):

LNPIRENLGRQWKNCARKLGFTESQIDEIDHD-
YERDGLKEKVYQMLQKWLMREGTKGATVGKLAQAL

One complete reading frame was identified which consisted of 116 EST's and covered 2715 bases. Translation of the contiguous sequence generated a putative protein sequence of 405 amino acids starting with a methionine. The putative protein contained three significant features: a transmembrane domain (amino acids 103-118), a death domain (amino acids 168-240) and a serine-proline rich region (amino acids 262-325).

Clone g1727750 which contains the 5′ sequence of DETH was obtained from the Merck/Washington University EST Project, St. Louis, Mo., USA and sequenced using an Applied Biosystems Model 377 Automated DNA Sequencer and standard fluorescent dye terminator chemistry. The cDNA clone g1727750 was made from mRNA of a human colon epithelial cell line cloned into a vector (Unizap, Stratagene, La Jolla, Calif., USA) at the EcoRI and XhoI sites and was in a 2.0 ml E. coli culture. The sequence of clone g1727750 is shown in SEQ ID NO: 1.

A pair of primers were designed to contain 5′ plus-strand sequence starting with the ATG (start codon) (Primer 1) and 3′ minus-strand sequence starting at the TAG (stop codon) (Primer 2) with Bgl II and Sal I restriction sites at the 5′ and 3′ ends, respectively. The primers were as follows:

| | |
|---|---|
| Primer 1 - | ttt aga tct atg aac tca aca gaa tcc aac tct tct gcc |
| Primer 2 - | gtc gac cta cag cag gtc agg aag atg gct ata aac ag |

The full length coding region (SEQ ID NO: 1 bases 190-1404) was then amplified by PCR with these primers and high-fidelity Vent DNA polymerase (New England Biolabs, Inc., Beverly, Mass., USA) using clone g1727750 plasmid DNA as the template in a 12 cycle PCR protocol (94° C. for one minute for denaturation, 62° C. for 30 seconds for annealing, 72° C. for 2 minutes for extension).

The 1.26 Kb PCR product was cut with Bgl II and Sal I, phenol-precipitated and ligated into plasmid pGEX6p-1 (Pharmacia Biotech Inc., Piscataway, N.J. USA at the Bam HI and Sal I sites. The resulting plasmid containing the 1.26 Kb PCR product was transformed into E. coli BL-21 (DE3)pLYSs cells (Stragagene, La Jolla, Calif., USA) and expressed as described in Example 4.

The PCR product was then sequenced using an Applied Biosystems Model 377 Automated DNA Sequencer and standard fluorescent dye terminator chemistry.

### Example 2

### Cloning of the Extracellular and Intracellular Regions of DETH

The extracellular region of DETH is comprised of amino acids 1-102, coded for by the 5′ 306 base pair (bp) portion of the DETH DNA sequence (SEQ ID NO: 1, bases 190-495). The intracellular region of DETH is comprised of amino acids 121-405 coded for by 855 base pairs of the DETH open reading frame (SEQ ID NO: 1, bases, bases 550-1404).

The extracellular and intracellular regions of the DETH protein were separately cloned using the PCR protocol described in Example 1 and the primers described below.

For the extracellular region the following primers containing the restriction sites Bgl II/Sal I were used:

| | |
|---|---|
| 5′ Bgl II for Primer 1 - | ttt aga tct atg aac tca aca gaa tcc aac tct tct gcc |
| 3′ EcoR I for Primer 3 - | ttt gaa ttc caa atg ctc att gat gtc aaa atg c |

For the intracellular region the following primers containing Bgl II/Eco RI restriction sites were used:

| | |
|---|---|
| 5′ Bgl II for Primer 4 - | ttt aga tct cgg aaa agc tcg agg act ctg aaa aag ggg |
| 3′ Sal I for Primer 2 - | gtc gac cta cag cag gtc agg aag atg gct ata aac ag |

The PCR product for the extracellular region was cut with Bgl II and Sal I and ligated into pGEX6p-1 at the Baam HI/Sal I site. The PCR product for the intracellular region was cut with Bgl II and Eco RI and ligated into pGEX6p-1 at the Bam HI/Eco RI site. The vector pGEX6p-1 (Pharmacia Biotech Inc., Piscataway, N.J.) creates a glutathione-S-transferase (GST) fusion protein which contains a glutathione recognition site.

The respective plasmids containing the extracellular region or the intracellular region were then amplified in accordance using the protocol described in Example 4 and sequenced.

### Example 3

### Expression of DETH in Human Cells

This DNA was ligated into pIRES-EGFP (Clontech, Palo Alto, Calif., USA) at the EcoR V site. Because the insert can be ligated in two (opposite) orientations, PCR was used to screen for the correct directionality. The recombinant DNA was transformed into E. coli and colonies were screened by a PCR method using a pair of primers, one of which was vector-specific and one that was DETH-specific. The size of the PCR product determined which plasmids contained correctly oriented inserts. Ten colonies were found carrying plasmids with the correct orientation of the full-length DETH sequence in pIRES-EGFP. Two of the ten plasmids were sequenced and found to be error free.

HeLa cells (American Type Culture Collection (ATCC) Accession number CCL-2, Rockville, Md., USA) grown in Dulbecco's Minimum Essential Medium (DMEM) supplemented with 10% fetal calf serum, 100 U/ml penicillin and 0.1 mg/ml streptomycin. A 549 cells (ATCC Accession Number CCL-185), a human lung carcinoma cell line, were grown in F-12K medium supplemented with 10% fetal calf serum, 100 U/ml penicillin and 0.1 mg/ml streptomycin. Cells were transfected by electroporation using 5-10 μg of plasmid DNA in 250 μL DMEM plus fetal calf serum and a Bio-Rad Gene Pulser II at the settings 0.22 Kr, 0.96 μF, 40-50 microseconds.

He La or A549 cells were split 24 hours prior to transfection. 2×10⁶ cells were transfected with pIRES containing the full length 1.2 Kb PCR product coding for DETH. 2×10⁶ cells were transfected with the pIkES vector alone and used as a negative control.

After cells were electroporated with equal amounts of DNA, they were seeded into a six-well plate with a cover slip in each well. At least 24 or 48 hours, the cover slips were picked and cells on them were fixed with 4% paraformaldehyde. With or without staining with the DNA stain 4,6-diamidino-2-phenylindole (DAPI) the coverslips were mounted on slides and examined by fluorescence microscopy. The percentage of green (fluorescent) cells from experimental samples were normalized according to the pIRES negative control. The cell survival rates were the summary of three independent experiments.

When DETH was overexpressed in He La cells, approximately 95% of cells containing the protein died by apoptosis. Apoptosis was verified by cell morphology. By contrast, there was nearly 100% survival of cells transfected with the pIRES vector only.

### 4. Expression of DETH and Regions of DETH as GST Fusion Proteins and Purification Thereof

The pGEX6p-1 plasmid constructs described in Examples 1 and 2, containing DNA coding for full-length DETH, the extracellular region, or the intracellular region, respectively, were transformed into E. coli BL-21 (DE3)pLysS cells (Stragagene, La Jolla, Calif., USA). Cells carrying these plasmids were grown in LB-AMP medium (Gibco BRL, Gaithersburg, Md., USA) to a density of A=0.5 as measured on a spectrophotometer at 600 nm. Isopropyl β-D-thiogalactoside (IPTG) was added to the cell cultures to a final concentration of 0.4 mM and the cells were incubated for two more hours. The cells were then harvested and resuspended in one tenth the culture volume of phosphate buffered saline (PBS) containing 100 μM/ml lysozyme and incubated on ice for thirty minutes to lyse the cells. The lysates were sonicated for one minute in the presence of 1% Nonidet p-40 (NP40) and 1.0 mM phenylmethylsulfonylfluoride (PMSF) and centrifuged at 15,000×g for forty minutes. The supernatants were poured over a 1 ml glutathione-Sepharose 4B column (Pharmacia) which was then washed with PBS and PreScission Cleavage Buffer (50 mM Tris-HCl pH 7.0, 150 mM NaCl, 1 mM EDTA, 1 mM dithiothreitol, Pharmacia). The DETH protein was cleaved from GST by a protease (Precission Protease, Pharmacia), 80 U/ml). The cleaved DETH proteins were checked by SDS-PAGE for purity.

The full-length GST-DETH fusion protein has been expressed in E. coli but was found to be insoluble. The cytoplasmic and extracellular regions of DETH were purified to approximately 99% purity.

In addition to the above purification procedure, an ion-exchange step using a Mono-S column (Pharmacia) with a linear 50-500 mM NaCl gradient has been successfully used to further purify the extracellular region.

### Example 5

### Polyclonal Antibodies

Soluble purified protein (recombinantly produced extracellular region or cytoplasmic region) were used to inject rabbits in accordance with the method in Antibody, A Laboratory Manual, Harlow and Lane, editors, Cold Spring Harbor Press, Cold Spring Harbor, N.Y., 1988.

### Example 6

### Northern Blot Analysis of Normal Human Tissues and Human Cancer Cell Lines

The 1.26 kb PCR product corresponding to the coding sequence of DETH was used to make a probe for northern blots. The PCR product was labeled using a random, hexanucleotide priming kit (Boehringer Mannheim, Indianapolis, Ind., USA) and gamma-P³²-dCTP (3,000 Ci/millimole, Amersham, Arlington Heights, Ill., USA). The procedure was carried out according to the manufacturer's instructions.

Northern analysis was done using a kit (Clontech Master Northern Blot, Clontech, Palo Alto, Calif., USA). Multiple tissue blots and tumor cell line blots used in the northern analysis were also purchased from Clontech. Northern analysis was performed in accordance with the manufacturer's instruction with the following modifications. Hybridizations were carried out at 68° C. for 2 hours in Expresshyb solution (Clontech). The blots were washed once each with two-fold concentrated SSPE buffer (Gibco/BRL, Gaithersburg, Md., USA), normal SSPE buffer and two-fold diluted SSPE buffer at 65° C. for 30 minutes. After the blots were dried, they were exposed to Kodak XAR 5 film with an intensifying screen at −80° C. for 24 hours. Alternatively, the blot membranes were quantified by use of a phosphoimager (Storm, phosphoImager, Molecular Dynamics) with an exposure of 2 hours. This gave a relative intensity_for each tissue or cell line.

The major message size found on this blot for DETH was approximately 4.0 Kb. The intensity of the signals are represented semi-quantitatively from absent (−) to very strong (+++).

| Cancer Cell Lines | Relative Intensity |
|---|---|
| HL-60, promyelocytic leukemia | − |
| Hela S3, cervical carcinoma | +/− |
| K-562, chronic myelogenous leukemia | − |
| MOLT-4, lymphoblastic leukemia | +/− |
| Raji, Burkitt's lymphoma | − |
| SW480, colorectal adenocarcinoma | + |
| A549, lung carcinoma | +++ |
| G361, melanoma | + |

The results show that the DETH message is strongly expressed in the lung cancer cell line A549 (more than 20-fold stronger than any other cell line), and more weakly expressed in melanoma and colorectal cancer cell lines.

| | Relative | | Relative |
|---|---|---|---|
| Tissue | Intensity | Tissue | Intensity |
| whole brain | +/− | amygdala | + |
| caudate nucleus | + | cerebellum | + |
| cerebral cortex | ++ | frontal lobe | ++ |
| hippocampus | ++ | medulla oblongata | +++ |
| occipital lobe | − | putamen | +/− |
| substantia nigra | ++ | temporal lobe | ++ |
| thalamus | ++++ | subthalamic nucleus | +++ |
| spinal cord | ++++ | heart | +/− |
| aorta | − | skeletal muscle | − |
| colon | + | bladder | ++ |
| uterus | ++ | prostate | ++ |
| stomach | +++ | testis | − |
| ovary | +/− | pancreas | +/− |
| pituitary gland | +/− | adrenal gland | +/− |
| thyroid gland | − | salivary gland | +/− |
| mammary gland | ++ | kidney | + |
| liver | − | small intestine | +/− |
| spleen | − | thymus | +/− |
| peripheral leukocyte | − | lymph node | +/− |
| bone marrow | − | appendix | + |
| lung | + | trachea | + |
| placenta | ++ | fetal brain | + |
| fetal heart | +/− | fetal kidney | ++ |
| fetal liver | − | fetal spleen | +/− |
| fetal thymus | +/− | fetal lung | + |

These data demonstrate that while low levels of DETH mRNA are found in many tissues, DETH is primarily a brain and, to a lesser extent, a stomach transcript.

1. A purified protein having an amino acid sequence having at least 80% identity to DETH.

2. The purified protein of claim 1 wherein said protein has an amino acid sequence having at least 90% identity to DETH.

3. The purified protein of claim 1 wherein said protein has an amino acid sequence having at least 95% identity to DETH.

4. The purified protein of claim 1 wherein said protein has an amino acid sequence having at least 99% identity to DETH.

5. The purified protein of claim 1 wherein said protein has the amino acid sequence of SEQ ID NO: 2.

6. A fragment of DETH that induces apoptosis.

7. A purified nucleic acid sequence encoding a protein of any one of claims 1-6.

8. An antibody specific for DETH.

9. A method of inducing apoptosis comprising the step of expressing DETH in a cell in an amount effective to induce apoptosis.
